(19) United States
(12) Patent Application Publication
(10) Pub. No.: US 2022/0309402 A1
(43) Pub. Date: Sep. 29, 2022

(54) METHOD, DEVICE AND MEDIUM FOR DATA PROCESSING

(71) Applicant: NEC CORPORATION, Tokyo (JP)

(72) Inventors: Wenjuan WEI, Beijing (CN); Lu Feng, Beijing (CN)

(73) Assignee: NEC CORPORATION, Tokyo (JP)

(21) Appl. No.: 17/702,277

(22) Filed: Mar. 23, 2022

(30) Foreign Application Priority Data
Mar. 23, 2021 (CN) 202110309510.7

(51) Int. Cl. G06N 20/00; G06N 5/04

(52) U.S. Cl. CPC G06N 20/00; G06N 5/04

(57) ABSTRACT

Embodiments of the present disclosure relate to method, device and computer-readable storage medium for data processing. A method for data processing comprises obtaining user data of a target user under a target environment. The user data comprises observational data of a plurality of features of the target user. The method further comprises extracting at least part of user data from the user data. The at least part of user data comprises observational data of at least one feature of the plurality of features which affects a target feature and has causal invariance. The method further comprises generating, based on the at least part of user data and a prediction model trained for the at least one feature, a prediction result for the target feature of the target user. The embodiments of the present disclosure further provide a device and a computer-readable storage medium that can perform the above method. The embodiments of the present disclosure can accurately and robustly make predictions based on features with causal invariance.

### TECHNICAL FIELD

Embodiments of the present disclosure relate to the field of machine learning, and more specifically, to method, apparatus and computer-readable storage medium for data processing.

### BACKGROUND

With the fast development of information technology, the scale of data has grown rapidly. Under such background and trend, machine learning has received more and more attention. Causal discovery thus has been widely applied in real life, such as in the fields of user service, healthcare and online advertising. The so-called causal discovery here refers to discovering causality between a plurality of features from sample data regarding the plurality of features. For example, in the user service field, results of causal discovery can be used to assist in understanding user satisfaction; in the healthcare field, results of causal discovery can be used to assist in understanding the recovery condition of patients; in the online advertising field, results of causal discovery can be used to assist in understanding users' interest in online advertising, etc.

### SUMMARY

Embodiments of the present disclosure provide a method, apparatus and computer-readable storage medium for data processing.

In a first aspect of the present disclosure, there is provided a method for data processing. The method comprises: obtaining a plurality of training datasets under a plurality of environments, each training dataset comprising observational data of a group of features of a user under a corresponding environment, the group of features comprising a target feature and a plurality of features related to the target feature; determining, based on the plurality of training datasets and invariance of causality under different environments, at least one feature that affects the target feature and has causal invariance from the plurality of features; and training a prediction model for the at least one feature by using at least one training dataset of the plurality of training datasets, the prediction model being used to generate a prediction result for the target feature of a target user under a target environment based on observational data of the at least one feature of the target user.

In a second aspect of the present disclosure, there is provided a method for data processing. The method comprises: obtaining user data of a target user under a target environment, the user data comprising observational data of a plurality of features of the target user; extracting at least part of user data from the user data, the at least part of user data comprising observational data of at least one feature of the plurality of features which affects a target feature and has causal invariance; and generating a prediction result for the target feature of the target user based on the at least part of user data.

In a third aspect of the present disclosure, there is provided an apparatus for data processing. The apparatus comprises: at least one processing unit; at least one memory, coupled to the at least one processing unit and storing instructions executed by the at least one processing unit, the instructions, when executed by the at least one processing unit, causing the apparatus to perform acts, the acts comprising: obtaining a plurality of training datasets under a plurality of environments, each training dataset comprising observational data of a group of features of a user under a corresponding environment, the group of features comprising a target feature and a plurality of features related to the target feature; determining, based on the plurality of training datasets and invariance of causality under different environments, at least one feature that affects the target feature and has causal invariance from the plurality of features; and training a prediction model for the at least one feature by using at least one training dataset of the plurality of training datasets, the prediction model being used to generate a prediction result for the target feature of a target user under a target environment based on observational data of the at least one feature of the target user.

In a fourth aspect of the present disclosure, there is provided is an apparatus for data processing. The apparatus comprises: at least one processing unit; at least one memory, coupled to the at least one processing unit and storing instructions executed by the at least one processing unit, the instructions, when executed by the at least one processing unit, causing the apparatus to perform acts, the acts comprising: obtaining user data of a target user under a target environment, the user data comprising observational data of the plurality of features of the target user; extracting at least part of user data from the user data, the at least part of user data comprising observational data of at least one feature of the plurality of features which affects a target feature and has causal invariance; and generating a prediction result for the target feature of the target user based on the at least part of user data.

In a fifth aspect of the present disclosure, there is provided a computer-readable storage medium. The computer-readable storage medium comprises computer-executable instructions stored thereon which, when being executed by a processor to perform the method according to the first aspect of the present disclosure.

In a sixth aspect of the present disclosure, there is provided a computer-readable storage medium. The computer-readable storage medium comprises computer-executable instructions stored thereon which, when being executed by a processor to perform the method according to the second aspect of the present disclosure.

This Summary is provided to introduce a selection of concepts in a simplified form that are further described below in the Detailed Description. This Summary is not intended to identify key features or essential features of the present disclosure, nor is it intended to be used to limit the scope of the present disclosure. Other features of the present disclosure will become easy to understand from the description below.

### BRIEF DESCRIPTION OF THE DRAWINGS

Through the following disclosure and claims, the objects, advantages and other features of the present invention will become more apparent. For the illustration purpose only, non-limiting description of preferable embodiments is provided with reference to the accompanying drawings, wherein:

FIG. 1 shows a schematic view of an example of a data processing environment in which some embodiments of the present disclosure can be implemented;

FIG. 2 shows a flowchart of an example method for training a prediction model according to embodiments of the present disclosure;

FIG. 3 shows a flowchart of an example method for using a prediction model according to embodiments of the present disclosure;

FIG. 4 shows a flowchart of an example method for predicting user satisfaction according to embodiments of the present disclosure;

FIG. 5 shows a flowchart of an example method for predicting the recovery condition of a patient according to embodiments of the present disclosure;

FIG. 6 shows a flowchart of an example method for predicting users' interest in online advertising according to embodiments of the present disclosure; and

FIG. 7 shows a schematic block diagram of an example computing device applicable to implement embodiments of the present disclosure.

Throughout the figures, the same or corresponding numerals denote the same or corresponding parts.

### DETAILED DESCRIPTION OF EMBODIMENTS

The embodiments will be described in more detail with reference to the accompanying drawings, in which some embodiments of the present disclosure have been illustrated. However, the present disclosure can be implemented in various manners, and thus should not be construed to be limited to embodiments disclosed herein. On the contrary, those embodiments are provided for the thorough and complete understanding of the present disclosure, and completely conveying the scope of the present disclosure to those skilled in the art. It is to be understood that the drawings and embodiments of the present disclosure are only used for illustration, rather than limiting the protection scope of the present disclosure.

The terms “comprise” and its variants used herein are to be read as open terms that mean “include, but is not limited to.” The term “based on” is to be read as “based at least in part on”. The term “one embodiment” or “the embodiment” is to be read as “at least one embodiment.” The terms “first,” “second” and the like may refer to different or the same objects. Other definitions, explicit and implicit, might be included below.

As discussed above, in real life, it is desirable to fast and accurately find causality between many features.

For example, in the user service field, operators may collect a large amount of user data (e.g., age, monthly consumption of Internet traffic, ratio of free traffic, total monthly consumption of Internet traffic of a user, etc.) in order to understand user satisfaction. Since the collected data might come from different environments (e.g., time, place, etc.), the collected data might not belong to the same distribution. In this case, if the collected data is assumed to come from the same distribution, then user satisfaction cannot be well predicted. In addition, operators might hope to understand the user satisfaction in a new environment. However, data distribution in the new environment might not belong to the same distribution as training data, thus the user satisfaction in the new environment cannot be well predicted.

Similarly, in the healthcare field, doctors may collect a large amount of patient data (e.g., gender, age, occupation, treatment plan of a patient, etc.) in order to understand the patient's recovery condition. Since the collected data might come from different environments (e.g., ages, genders, etc.), data might not belong to the same distribution. In this case, if the collected data is assumed to come from the same distribution, then the patient's recovery cannot be well predicted. In addition, doctors might hope to understand the patient's recovery in a new environment. However, data distribution in the new environment might not belong to the same distribution as training data, thus the patient's recovery in the new environment cannot be well predicted.

Further, in the online advertising field, advertising providers may collect a large amount of user data (e.g., gender, age, occupation of a user, etc.) and a large amount of online advertising data (e.g., size, duration, display position, content, quality of an online advertisement, etc.) in order to understand users' interest in online advertising. Since data being collected might come from different environments (e.g., ages, genders, regions, etc.), data might not belong to the same distribution. In this case, if collected data is assumed to come from the same distribution, then the user's interest in online advertising cannot be well predicted. In addition, advertising operators might hope to understand the user's interest in online advertising in a new environment. However, data distribution in the new environment might not belong to the same distribution as training data, thus the user's interest in online advertising in the new environment cannot be well predicted.

The embodiments of the present disclosure propose a solution for data processing to solve one or more of the above and/or other potential problems. In the solution, features with causal invariance that affect target features in different environments can be found, and a prediction model is trained for these features, so that target features can be accurately predicted in new environments according to the trained prediction model.

The various embodiments of the present disclosure will be described in detail in conjunction with an example scenario in the user service field. It is to be understood this is merely for the illustration purpose but not intended to limit the scope of the present invention in any way.

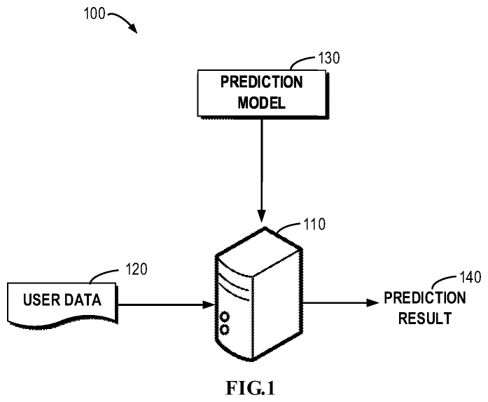

FIG. 1 shows an example schematic view of a data processing environment 100 in which some embodiments of the present disclosure can be implemented. The environment 100 comprises a computing device 110. The computing device 110 may be any device with computing capability, such as a personal computer, a tablet computer, a wearable device, a cloud server, a mainframe, a distributed computing system, etc.

The computing device 110 may obtain user data 120 of a target user under a target environment. The computing device 110 may use a trained prediction model 130 to generate a prediction result 140 (e.g., satisfied or dissatisfied, what is the satisfaction) for a target feature (e.g., user satisfaction) of the target user based on the user data 120.

The trained prediction model 130 may generate a prediction result 140 based on observational data of at least one feature with causal invariance that affects the target feature in the user data 120. Features with causal invariance refer to such features whose distribution under different environments will remain unchanged given observational data of these features. That is, if features have causal invariance under different environments, then the impact of these features on the target feature under different environments is consistent. Thus, given the observational data of these features, the target feature under different environments belongs to a same distribution.

In view of this, compared with all user data 120 that might comprise observational data of a feature without causal invariance, using observational data of at least one feature with causal invariance may obtain a more accurate prediction result.

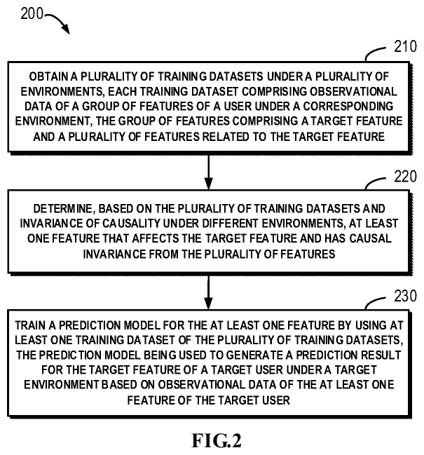

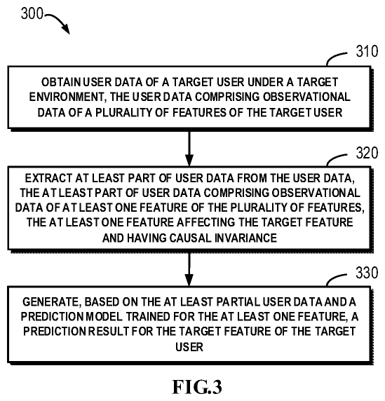

Description on how to determine a feature that affects a target feature and has causal invariance and how to train the prediction model 130 will be described with reference to FIG. 2, and description on how to use the trained prediction model 130 will be described with reference to FIG. 3.

FIG. 2 shows a flowchart of an example method 200 for training the prediction model 130 according to the embodiments of the present disclosure. For example, the method 200 may be performed by the computing device 110 as shown in FIG. 1. It is to be understood that the method 200 may further comprise additional blocks which are not shown and/or may omit some blocks which are shown. The scope of the present disclosure is not limited in this regard.

At block 210, the computing device 110 obtains a plurality of training datasets under a plurality of environments. The plurality of environments may be regarded as a plurality of groups under specific classifications. The specific classifications may be determined based on an application scenario. For example, the plurality of environments may be various groups under geographical classifications (e.g., Beijing, Shanghai, etc.), various groups under age group classifications (e.g., youth age group, middle age group, old age group, etc.), or various groups under data obtaining time classifications (e.g., January, February, etc.). Each training dataset comprises observational data of a group of features of a user under a corresponding environment. The group of features comprises a target feature and a plurality of features related to the target feature.

For example, in an example scenario in the user service field, suppose the plurality of environments is a plurality of regions. In this case, one training dataset may comprise observational data of a group of features of users in Beijing, and another training dataset may comprise observational data of a group of features of users in Shanghai, and so on and so forth.

In addition, suppose the plurality of environments is a plurality of age groups. In this case, one training dataset may comprise observational data of a group of features of users in youth age group (e.g., 18 to 30 years old), another training dataset may comprise observational data of a group of features of users in middle age group (e.g., 30 to 60 years old), a further training dataset may comprise observational data of a group of features of users in old age group (e.g., over 60 years old), and so on and so forth.

Further, suppose the plurality of environments is a plurality of data obtaining times. In this case, one training dataset may comprise observational data of a group of features of users obtained in January; another training dataset may comprise observational data of a group of users obtained in February, and so on and so forth.

In some embodiments, a group of features of users may comprise user behavior features and user satisfaction features, etc. As an example, user behavior features may comprise user attribute features (such as gender, age, grade of a user, etc.), package features (such as package name, package charges, package traffic, etc.), monthly consumption features (such as calling/called call duration, the number of calling/called calls, free traffic usage, application traffic usage, supplementary traffic times, etc.), monthly charge features (such as voice charges, out-of-package voice charges, traffic charges, international roaming traffic charges, etc.), and/or service features (such as the number of customer service requests, the number of account logins, the number of business transactions, the number of complaints, etc.), etc. In addition, user behavior features may further comprise user text information features (such as comments, content of complaints of the user, etc.), and/or web browsing information features, etc.

Further, as an example, user satisfaction features may comprise user overall satisfaction, charge satisfaction, network quality satisfaction, voice call quality satisfaction, business promotion satisfaction, business transaction satisfaction, business hall service satisfaction, aspects to be improved, and/or aspects of satisfaction, etc.

Therefore, observational data of a group of features may be values of the above features.

In some embodiments, to obtain the plurality of training datasets, the computing device 110 may collect observational data of the group of features of users from the plurality of environments. The computing device 110 may group the collected observational data based on environment parameters identifying different environments to obtain the plurality of training datasets corresponding to the plurality of environments.

For example, as described above, observational data of a group of features of users from a plurality of regions (e.g., Beijing, Shanghai, etc.) may be collected, and the collected observational data may be grouped based on different regions to obtain a plurality of training datasets corresponding to the plurality of regions. Also, observational data of a group of features of users from a plurality of age groups (e.g., youth age group, middle age group, old age group, etc.) may be collected, and the collected observational data may be grouped based on different age groups to obtain a plurality of training datasets corresponding to the plurality of age groups. Further, observational data of a group of features of users from the plurality of data obtaining times (e.g., January, February, etc.) may be collected, and the collected observational data may be grouped based on different data obtaining times to obtain a plurality of training datasets corresponding to the plurality of data obtaining times.

Further, in some embodiments, the computing device 110 may perform preprocessing, feature engineering, and/or feature selection on the plurality of training datasets to enhance the plurality of training datasets. For example, during the preprocessing, the computing device 110 may obtain, based on a package name, a new feature indicating whether a package is an unlimited traffic package. For another example, the computing device 110 may obtain, based on content of a complaint, new features indicating whether the complaint is a complaint for charges, a complaint for service, a complaint for network quality, etc. Further, the computing device 110 may obtain, based on properties of words in observational data of content of complaints (e.g., text of content of complaints), observational data of these new features, such as numerical representations between 0 and 100, wherein 0 represents no complaint, and 100 represents extreme dissatisfaction. As a further example, the computing device 110 may obtain a new feature indicating the number of traffic queries based on the web browsing information feature.

In some embodiments, during the feature engineering, the computing device 110 may process existing features to generate new features indicating new properties (e.g., proportions, marginal ratios, etc.). For example, these features may comprise voice charge proportion (which is voice charges divided by total charges), proportion of calling calls (which is calling calls divided by total calls), and/or voice marginal ratio (which is calling call duration divided by voice charges), etc. In addition, or alternatively, the computing device 110 may further process periodical features to generate new features indicating new properties (e.g., mean, variance, fluctuation, etc.) within a certain period of time. For example, these features may comprise average voice charges (which is 0.5*(voice charges of the previous month+voice charges of the previous two months)), and/or the fluctuation of voice charge proportion (which is the voice charge proportion of the previous month−the voice charge proportion of the previous two months), etc.

In some embodiments, the features may be filtered to select features related to a target feature (e.g., user satisfaction). During the feature selecting, the computing device 110 may use a Lasso (least absolute shrinkage and selection operation) algorithm, a Random Forest algorithm and other feature selecting method to select features related to the target feature.

At block 220, the computing device 110 determines, based on the plurality of training datasets and according to invariance of causality under different environments, at least one feature from the plurality of features. The at least one feature affects the target feature and has causal invariance.

As described above, features having causal invariance refer to such features that given observational data of these features under different environments, the distribution of the target feature will remain unchanged. That is, if features have causal invariance under different environments, then given observational data of these features, the target feature belongs to the same distribution under different environments. Suppose package features can affect the target feature and have causal invariance, while monthly charge features cannot affect the target feature and/or do not have causal invariance, then the at least one feature will comprise package features but not comprise monthly charge features.

In some embodiments, to determine the at least one feature from the plurality of features, the computing device 110 may utilize various causal techniques, e.g., causal migration learning techniques, invariant causal prediction (ICP) techniques, etc.

At block 230, the computing device 110 trains a prediction model for the at least one feature by using at least one training data set of the plurality of training datasets. The prediction model is used to generate a prediction result for the target feature of a target user based on observational data of the at least one feature of the target user under a target environment.

The prediction model is trained with respect to features with causal invariance, so that the prediction model can generate a prediction result for the target feature of a target user based on observational data of features with causal invariance of the target user under a target environment.

In some embodiments, the prediction model may indicate one of linear causality and nonlinear causality between the at least one feature and the target feature. For example, depending on whether there is linear causality or nonlinear causality between the at least one feature and the target feature, the prediction model may be linear or nonlinear.

In some embodiments, to train the prediction model, the computing device 110 may obtain a group of training samples from the at least one training dataset. Each training sample comprises observational data of the at least one feature of a corresponding user and observational data of the target feature. For example, as described above, suppose that the package feature can affect the target feature and has causal invariance, and then a training sample may be observational data of the package feature of a corresponding user and observational data of the user satisfaction.

Thereby, the computing device 110 may train the prediction model according to a machine learning algorithm and based on the group of training samples. The machine learning algorithm may be any appropriate machine learning algorithm, e.g., K-nearest neighbor, SVM (support vector machine) algorithm, etc. In this way, since the prediction model is trained using observational data of features with causal invariance under different environments, the trained prediction model may obtain a more accurate prediction result under a target environment.

In addition, in some embodiments, to train the prediction model based on the group of training samples, the computing device 110 may determine a transformation manner for performing data transformation on each training sample in the group of training samples. The transformation manner may be determined based on various appropriate algorithms, e.g., kernel-based optimization algorithms such as DICA (domain-invariant component analysis) algorithm, SCA (scatter component analysis) algorithm, etc. The kernel-based optimization algorithm may learn different transformations by minimizing cross-domain differences while preserving functional relationships between input and output variables. In such case, the transformed training samples may have independent identical distributions. Therefore, the computing device 110 may obtain a group of transformed training samples based on the transformation manner and train the prediction model based on the group of transformed training samples.

Further, in some embodiments, the computing device 110 may separately train respective prediction models for different environmental classifications. For example, the computing device 110 may separately train respective prediction models for the geographical regions, the age groups and the data obtaining times. The trained prediction models and corresponding environment information may be stored in a storage device.

FIG. 3 shows a flowchart of an example method 300 for using the prediction model according to the embodiments of the present disclosure. For example, the method 300 may be performed by the computing device 110 as shown in FIG. 1. It is to be understood that the method 300 may further comprise additional blocks not shown and/or may omit some blocks which are shown. The scope of the present disclosure is not limited in this regard.

At block 310, the computing device 110 obtains user data 120 of a target user under a target environment. The user data 120 comprises observational data of a plurality of features of the target user. The user data 120 comprises but not limited to at least one of user behavior data of product or service usage, attribute data and research data. For example, in an example scenario of the user service field, the plurality of features of the target user may comprise a behavior feature of the target user. An example of the behavior feature has been described above, and thus the detailed description is omitted here. The observational data of the plurality of features may be values of the above features.

At block 320, the computing device 110 extracts at least part of user data from the user data 120. The at least part of user data comprises observational data of at least one feature of the plurality of features which affects a target feature and has causal invariance. As an example, in an example scenario of the user service field, the target feature may be the user satisfaction. An example of the user satisfaction has been described above, and thus the detailed description is omitted here. A prediction result of the target feature may be a predicted value of the target feature.

As described above, features with causal invariance refer to such features that under different environments, given observational data of these features, the distribution of the target feature will keep unchanged. That is, if features have causal invariance under different environments, then given observational data of these features, the target feature belongs to the same distribution under different environments. Suppose that the package feature can affect the target feature and has causal invariance, while the monthly charge feature does not affect the target feature or does not have causal invariance, then the at least one packet comprises the package feature but does not comprise the monthly charge feature.

At block 330, the computing device 110 generates a prediction result 140 for the target feature of the target user based on the at least part of user data.

The prediction model has been described as having been trained for features with causal invariance under different environments. Since these features have causal invariance under different environments, they also have causal invariance under the target environment. In this case, the trained prediction model may accurately predict a prediction result of the target feature under the target environment based on observational data of features with causal invariance. Thereby, in some embodiments, the computing device 110 generates, based on the at least part of user data and according to the prediction model trained for the at least one feature, the prediction result 140 for the target feature of the target user.

Further, in some embodiments, the computing device 110 may determine the target environment from the plurality of environments. In some embodiments, the target environment may be automatically determined by the computing device 110 or manually selected by a user. For example, in an example scenario of the user service field, the user may select a desired target environment. For example, if the user wants to predict the user satisfaction in Shenzhen, then the user may input or select Shenzhen as the target environment. In such case, since respective prediction models are trained for different environment classifications, the computing device 110 may receive the input target environment information and determine, based on the target environment, a prediction model corresponding to the classification of the target environment. For example, suppose that respective prediction models are trained for the geographical regions, the age groups and the data obtaining times, since the target environment selected by the user belongs to the geographical region classification, the computing device 110 may select a prediction model corresponding to a geographical region.

Thereby, the accuracy of the prediction result may be increased under different environment classifications. In addition, since the user may select the target environment, the system flexibility and the user experience may be improved.

In some embodiments, the prediction result 140 may be used for subsequent analysis. For example, in the user service field, prediction results of the user satisfaction can be used by operators to adopt different policies for different users to improve the user satisfaction. In the field of health care, prediction results of the recovery conditions of patients can be used by doctors to formulate different medical plans for different patients to improve the cure rate. In the field of online advertising, users' interest in online advertising can be used by advertising providers to deliver different advertisements to different users to increase advertising revenue.

To this end, in some embodiments, the method 300 may further comprise outputting first information or performing a first operation based on the prediction result. The first information may comprise but not limited to one or more of indication information, policy information and recommendation information determined based on the prediction result 140. The first operation may comprise but not limited to performing a policy instruction operation, an identification operation, an analysis operation and the like based on the prediction result.

In addition, data generated by a subsequent act taken based on the prediction result 140 may further be used to improve the prediction model 130. Therefore, the accuracy of the prediction result may further be increased, and the prediction model may be caused to be dynamically updated. To this end, in some embodiments, the computing device 110 may obtain data generated by a subsequent act taken based on the prediction result 140 and update the prediction model 130 based on such data.

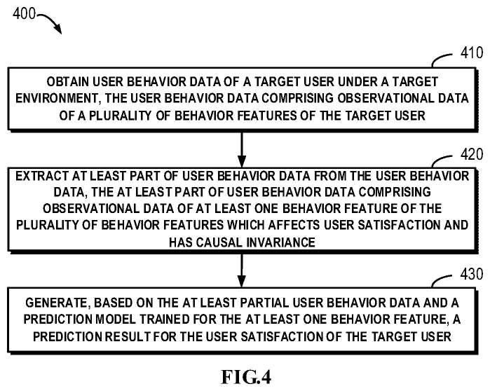

FIG. 4 shows a flowchart of an example method 400 for predicting user satisfaction according to the embodiments of the present disclosure. For example, the method 400 may be performed by the computing device 110 as shown in FIG. 1. It is to be understood that the method 400 may further comprise additional blocks not shown and/or may omit some blocks which are shown. The scope of the present disclosure is not limited in this regard.

At block 410, the computing device 110 may obtain user data of a target user under a target environment (e.g., a target region such as Shenzhen). The user data may comprise observational data of a plurality of behavior features of the target user. An example of the behavior feature has been described above, and thus the detailed description is omitted here. The observational data of the plurality of behavior features may be values of the above features.

At block 420, the computing device 110 may extract at least part of user behavior data from the user data. The at least part of user behavior data may comprise observational data of at least one behavior feature of the plurality of behavior features which affects user satisfaction and has causal invariance.

At block 430, the computing device 110 may generate a prediction result for the user satisfaction of the target user based on the at least part of behavior user data. Therefore, the accuracy of the predicted user satisfaction may be increased.

The method 400 may further comprise determining policy information for the one or more target users by using the prediction result of the user satisfaction. The method 400 may further comprise outputting the policy information or performing a policy operation based on policy information.

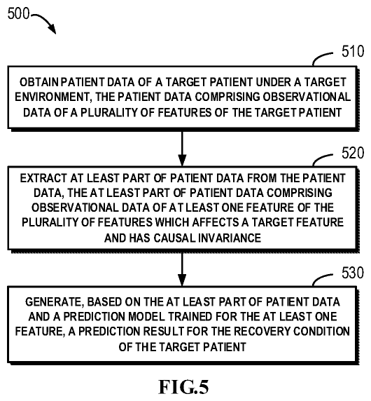

FIG. 5 shows a flowchart 500 of an example method for predicting the recovery condition of a patient according to the embodiments of the present disclosure. For example, the method 500 may be performed by the computing device 110 as shown in FIG. 1. It is to be understood that the method 500 may further comprise additional blocks not shown and/or may omit some blocks which are shown. The scope of the present disclosure is not limited in this regard.

At block 510, the computing device 110 may obtain patient data of a target patient under a target environment (e.g., a target age group such as child age group). The patient data may comprise observational data of a plurality of features of the target patient. For example, the plurality of features may comprise the patient's gender, region, treatment plan, etc. The observational data of the plurality of features may be values of the above features.

At block 520, the computing device 110 may extract at least part of patient data from the patient data. The at least part of patient data may comprise observational data of at least one feature of the plurality of features which affects the recovery condition of the patient and has causal invariance.

At block 530, the computing device 110 may generate a prediction result for the recovery condition of the target patient based on the at least part of patient data. Therefore, the accuracy of the predicted recovery condition of the patient may be increased.

The method 500 may further comprise determining treatment plan information or adjuvant treatment information for the one or more target patients by using the prediction result of the recovery condition of the target patient. The method 500 may further comprise outputting the treatment plan information or the adjuvant treatment information. In addition, the method 500 may further comprise making subsequent analysis on the treatment plan information or the adjuvant treatment information. Thereby, it is possible to assist doctors in making decisions about the treatment plan for the one or more target patients or treat the one or more target patients.

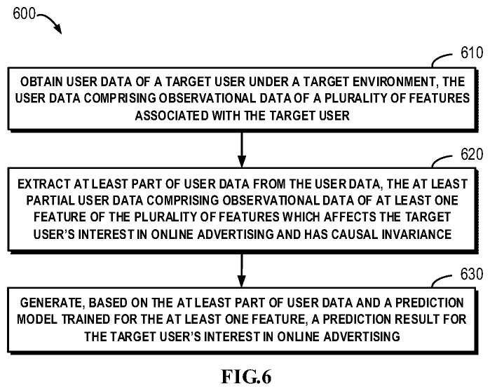

FIG. 6 shows a flowchart 600 of an example method for predicting a user's interest in online advertising according to the embodiments of the present disclosure. For example, the method 600 may be performed by the computing device 110 as shown in FIG. 1. It is to be understood that the method 600 may further comprise additional blocks not shown and/or may omit some blocks which are shown. The scope of the present disclosure is not limited in this regard.

At block 610, the computing device 110 may obtain user data of a target user under a target environment (e.g., a target gender such as female). The user data may comprise observational data of a plurality of features associated with the target user. For example, the plurality of features may comprise the user's age, occupation and region, as well as the size, duration, display location, content and quality of online advertising watched by the user. The observational data of the plurality of features may be values of the above features.

At block 620, the computing device 110 may extract at least part of user data from the user data. The at least part of user data may comprise observational data of at least one feature of the plurality of features which affects the user's interest in online advertising and has causal invariance.

At block 630, the computing device 110 may generate a prediction result for the target user's interest in online advertising based on the at least part of user data. Therefore, the accuracy of the user's interest in online advertising may be increased.

The method 600 may further comprise determining online advertising recommendation policy information for the one or more target users by using the prediction result of the user's interest in online advertising, or determining online advertising to be recommended to the one or more target users. The method 600 may further comprise outputting online advertising recommendation policy information, or recommending online advertising based on the online advertising recommendation policy information. In addition, the method 600 may further comprise presenting the recommended online advertising to the one or more target users.

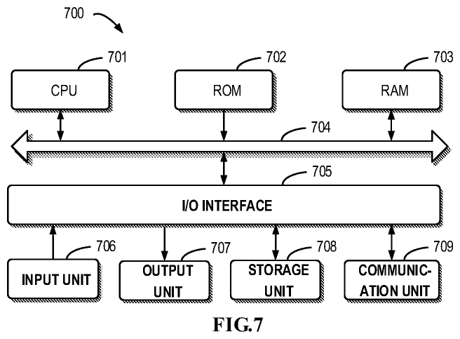

FIG. 7 shows a schematic block diagram of an example device 700 suitable for implementing embodiments of the present disclosure. For example, the computing device 110 as shown in FIG. 1 may be implemented by the device 700. As depicted, the device 700 comprises a central processing unit (CPU) 701 which is capable of performing various appropriate actions and processes in accordance with computer program instructions stored in a read only memory (ROM) 702 or computer program instructions loaded from a storage unit 708 to a random access memory (RAM) 703. In the RAM 703, there are also stored various programs and data required by the device 700 when operating. The CPU 701, the ROM 702 and the RAM 703 are connected to one another via a bus 704. An input/output (I/O) interface 705 is also connected to the bus 704.

A plurality of components in the device 700 are connected to the I/O interface 705, comprising: an input unit 706 such as a keyboard, a mouse, or the like; an output unit 707, such as various types of displays, a loudspeaker or the like; a storage unit 708, such as a disk, an optical disk or the like; and a communication unit 709, such as a LAN card, a modem, a wireless communication transceiver or the like. The communication unit 709 allows the device 700 to exchange information/data with other device via a computer network, such as the Internet, and/or various telecommunication networks.

The above-described procedures and processes, such as the methods 200, 300, 400, 500 and/or 600, may be executed by the processing unit 701. For example, in some embodiments, the methods 200, 300, 400, 500 and/or 600 may be implemented as a computer software program, which is tangibly embodied on a machine readable medium, e.g. the storage unit 708. In some embodiments, part or the entirety of the computer program may be loaded to and/or installed on the device 700 via the ROM 702 and/or the communication unit 709. The computer program, when loaded to the RAM 703 and executed by the CPU 701, may execute one or more acts of the methods 200, 300, 400, 500 and/or 600 as described above.

The embodiments of the present disclosure may be implemented as a system, device, method, and/or a computer program product. The computer program product may comprise computer-readable storage medium which stores computer-readable program instructions thereon to perform various aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the present disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the figures illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It is also to be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The descriptions of the various embodiments of the present disclosure have been presented for purposes of illustration, but are not intended to be exhaustive or limited to embodiments disclosed. Many modifications and variations will be apparent to those of ordinary skill in the art without departing from the scope and spirit of the described embodiments. The terminology used herein was chosen to best explain the principles of embodiments, the practical application or technical improvement over technologies found in the marketplace, or to enable others of ordinary skill in the art to understand embodiments disclosed herein.

1.-14. (canceled)

15. A method for data processing, comprising:
obtaining a plurality of training datasets under a plurality of environments, each of the training datasets comprising observational data of a group of features of a user under a corresponding environment, the group of features comprising a target feature and a plurality of features related to the target feature;
determining, based on the plurality of training datasets and invariance of causality under different environments, at least one feature that affects the target feature and has causal invariance from the plurality of features; and
training a prediction model for the at least one feature by using at least one training dataset of the plurality of training datasets, the prediction model being used to generate a prediction result for the target feature of a target user under a target environment based on observational data of the at least one feature of the target user.

16. The method according to claim 15, wherein obtaining the plurality of training datasets comprises:
collecting observational data of the group of features of users from the plurality of environments; and
grouping the collected observational data based on environment parameters identifying different environments to obtain the plurality of training datasets corresponding to the plurality of environments.

17. The method according to claim 15, wherein determining the at least one feature comprises:
determining the at least one feature from the plurality of features by using causal migration learning technique.

18. The method according to claim 15, wherein determining the at least one feature comprises:
determining the at least one feature from the plurality of features by using invariant causal prediction technique.

19. The method according to claim 15, wherein training the prediction model comprises:
obtaining a group of training samples from the at least one training data set, each training sample comprising observational data of the at least one feature of a corresponding user and observational data of the target feature; and
training the prediction model based on the group of training samples and using a machine learning algorithm.

20. The method according to claim 19, wherein training the prediction model based on the group of training samples comprises:
determining a transformation manner for performing data transformation on each training sample in the group of training samples;
obtaining a group of transformed training samples based on the transformation manner; and
training the prediction model based on the group of transformed training samples.

21. The method according to claim 15, further comprising:
obtaining user data of the target user under the target environment, the user data comprising observational data of a plurality of features of the target user;
extracting at least part of user data from the user data, the at least part of user data comprising observational data of at least one feature of the plurality of features, the at least one feature affecting a target feature and having causal invariance; and
generating a prediction result for the target feature of the target user based on the at least part of user data.

22. The method according to claim 21, further comprising:
determining the target environment from a plurality of environments.

23. The method according to claim 21, further comprising:
determining, based on the target environment, a prediction model for generating the prediction result from one or more prediction models.

24. The method according to claim 21, wherein generating the prediction result comprises:
generating, based on the at least part of user data and a prediction model trained for the at least one feature, a prediction result for the target feature of the target user.

25. An apparatus for data processing, comprising:
at least one processing unit;
at least one memory, coupled to the at least one processing unit and storing instructions executed by the at least one processing unit, the instructions, when executed by the at least one processing unit, causing the apparatus to perform the method according to claim 1.

26. A computer-readable storage medium, having computer-executable instructions stored thereon which, when executed by a device, causing the device to perform the method according to claim 1.
